(19) United States
(12) Patent Application Publication
(10) Pub. No.: US 2012/0109280 A1
(43) Pub. Date: May 3, 2012

(54) Torsion Constrained Stent Delivery System

(75) Inventor: Vincent McHugo, Birdhill (IE)

(73) Assignee: Cook Medical Technologies LLC, Bloomington, IN (US)

(21) Appl. No.: 13/278,563

(22) Filed: Oct. 21, 2011

Related U.S. Application Data
(60) Provisional application No. 61/407,635, filed on Oct. 28, 2010.

(51) Int. Cl. A61F 2/84

(52) U.S. Cl. 623/1.12

(57) ABSTRACT

A stent delivery system and a method are provided. The stent delivery system includes an inner and outer elongate shaft. The outer shaft is coaxially positioned over at least a portion of the inner shaft and the inner shaft is rotatably positionable relative to the outer shaft about a longitudinal axis from a first to a second rotational position. The stent delivery system also includes a stent positioned on at least a portion of the inner shaft and having a constrained and an expanded configuration. Proximal and distal constraining members releasably connected to the stent. The proximal and the distal constraining members cooperatively apply a torsional force to at least a portion of the stent in the constrained configuration and the inner and outer shafts in the first rotational position.

### RELATED APPLICATIONS

This application claims the benefit of U.S. Provisional Application No. 61/407,635, filed Oct. 28, 2010, which is incorporated by reference herein in its entirety.

### TECHNICAL FIELD

This invention relates to a medical device and, in particular to a device for delivering and deploying a stent and a method of delivering and deploying the stent into a body lumen.

### BACKGROUND

A self-expanding stent is typically introduced into the body using a delivery device that includes an outer sheath coaxially disposed and slidable over an inner catheter. The stent is disposed at the distal end of the device between the inner catheter and the outer sheath and held in a compressed position by the outer sheath. The inner catheter and the outer sheath move coaxially with respect to each other. The stent may be deployed by proximally pulling back the outer sheath relative to the inner catheter until the stent is exposed. The self-expanding stent expands from the stent distal end to the stent proximal end as the sheath is proximally withdrawn.

Several problems may occur with the sheathed delivery device described above. The sheath release delivery devices are difficult to reposition or remove and slow to operate. The stent may only be partially deployed prior to reconstrainment of the stent by the sheath in order to still reposition or remove the stent. Once the stent is fully deployed, i.e. radially expanded, the sheath cannot reconstrain the stent. For example, utilizing a conventional outer sheath/inner catheter delivery device may cause the physician to inadvertently use excessive force and pull back the outer sheath too far, thereby prematurely deploying the stent in an incorrect position within a body lumen. At this step in the procedure, repositioning of the stent becomes difficult, if not impossible, because the stent has already radially self-expanded into the body lumen. Additionally, retraction of the outer sheath may not be achieved with controlled movement because the physician is manually retracting the outer sheath which may lead to uneven or inadvertent jerking back of the outer sheath that can lead to improper positioning of the stent.

In a typical sheath release device where the outer sheath is proximally withdrawn, the first portion of the self-expanding stent to make contact with the body vessel is the most distal portion of the stent. This type of release may cause difficulty in accurately placing the proximal portion of the stent because the distal end of the stent is positioned first while the proximal portion of the stent is still covered by the outer sheath. Similarly, the positioning of the stent body in the central portion of the stent may be difficult to accurately position with a distal stent release system. Accurate placement of the proximal portion of the stent and/or the stent body may be important in certain applications, for example to prevent stent migration or to properly open a stricture along the entire length of the stricture. An additional drawback occurs with the sheathed stent delivery system where direct visualization of the stent is required. For example, in endoscopically placed stents, the sheath tends to prevent or obscure the location of the stent, making accurate placement of the stent more difficult.

Further potential drawbacks for the conventional sheathed stent delivery system involve the stent placement within the system prior to use within a patient. Loading and anchoring of a conventional sheathed stent delivery device is an involved process that may require preloading the stent into the device so that the stent remains compressed within the sheath during shipment and storage prior to use in the patient. Extended compression of the stent may lead to an alteration in the stent mechanical properties.

Conventional sheathed stent delivery devices also require a high force to overcome the friction between the stent and the sheath that may also be a problem for proper stent placement within the patient. The introducer must be mechanically stronger to overcome the frictional forces to avoid undesirable frictional consequences such as stretching of the introducer catchers and hysterics in the movement of the stent. The sheathed stent delivery device also requires more space within an endoscope compared to a sheathless device and also adds additional expense to the delivery system.

Accordingly, in view of the drawbacks of current technology, there is a desire for a delivery system that can increase the control, accuracy and ease of placement of a stent during deployment of the stent within a patient. The delivery system would ideally reduce the risk of malfunction while providing for a smoother, more accurate and quicker deployment of the entire stent. The delivery system also would provide the ability to reconstrain, recapture, reposition and/or remove the stent after expansion of the stent.

### SUMMARY OF THE INVENTION

Accordingly, it is an object of the present invention to provide a device and a method having features that resolve or improve on one or more of the above-described drawbacks.

The foregoing object is obtained in one aspect of the present invention by providing a stent delivery system. The stent delivery system includes an inner elongate shaft including a proximal portion, a distal portion, a lumen extending at least partially therethrough, and an outer elongate shaft including a proximal portion and a distal portion. The outer elongate shaft is coaxially positioned over at least a portion of the inner elongate shaft and the inner shaft is rotatably positionable relative to the outer shaft about a longitudinal axis from a first rotation position to a second rotation position. The stent delivery system also includes a stent having a proximal portion and a distal portion, the stent positioned on at least a portion of the inner elongate shaft and having a constrained configuration and an expanded configuration. A proximal constraining member and a distal constraining member releasably connected to the stent. The proximal constraining member and the distal constraining member cooperatively apply a torsional force to at least a portion of the stent in the constrained configuration and the inner and outer elongate shafts in the first rotational position.

In another aspect of the present invention, a method for implanting a stent using a stent delivery system is provided. The method includes inserting a distal portion of a stent delivery system into the lumen of a patient. The stent delivery system includes an inner shaft including a proximal portion, a distal portion, a lumen extending at least partially therethrough, and an outer shaft including a proximal portion, a distal portion where the outer elongate shaft is coaxially positioned over at least a portion of the inner elongate shaft. The stent delivery system further includes a stent having a proximal portion and a distal portion, the stent positioned on at least a portion of the inner elongate shaft and a proximal constraining member releasably connected to the stent proximal portion and a distal constraining member releasably connected to the stent distal portion. The method further includes holding the stent in a constrained configuration with torsional force applied to the stent by the proximal and distal constraining members and the inner and outer elongate shafts in the first rotational position, positioning the stent at the implant site and expanding the stent to an expanded configuration by rotating the inner shaft relative to the outer shaft from the first rotational position to a second rotational position and releasing torsional force on the stent.

### BRIEF DESCRIPTION OF THE DRAWINGS

FIG. 1 is a sectional view of a stent delivery system according to an embodiment of the present invention showing a stent in a constrained configuration;

FIG. 2 is a sectional view of the device shown in FIG. 1 showing the stent in an expanded configuration;

FIG. 3 is a partial view of the device shown in FIG. 2 illustrating rotation;

FIG. 4A is a partial view of a stent in an expanded configuration;

FIG. 4B is a partial view of the stent shown in FIG. 4A in a constrained configuration;

FIG. 4C is a partial view of a stent in an expanded configuration;

FIG. 4D is a partial view of the stent shown in FIG. 4C in a constrained configuration;

FIG. 5 is a sectional view of a delivery system illustrating an outer sheath;

FIG. 6A is a partial perspective view of an embodiment of a proximal constraining member;

FIG. 6B is a partial side view of an embodiment of a distal constraining member;

FIG. 7 is a perspective view of an alternative embodiment of a constraining member;

FIG. 8A is a partial side view of an alternative embodiment of a distal constraining member;

FIG. 8B is a partial side view of an alternative embodiment of a distal constraining member;

FIG. 9 is a sectional view of an embodiment of a delivery system illustrating a stiffening member;

FIG. 10 is a sectional view of an alternative embodiment of a delivery system according to the present invention;

FIG. 11 is a sectional view of the device shown in FIG. 10 with a proximal outer sheath withdrawn and the stent in a constrained configuration;

FIG. 12 is a sectional view of the device shown in FIG. 11 with the stent in an expanded configuration; and

FIGS. 13A-13G illustrate alternative embodiments of a distal constraining member.

### DETAILED DESCRIPTION OF THE EMBODIMENTS

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

As used in the specification, the terms proximal and distal should be understood as being in the terms of a physician delivering the stent to a patient. Hence the term “distal” means the portion of the delivery system that is farthest from the physician and the term “proximal” means the portion of the delivery system that is nearest to the physician.

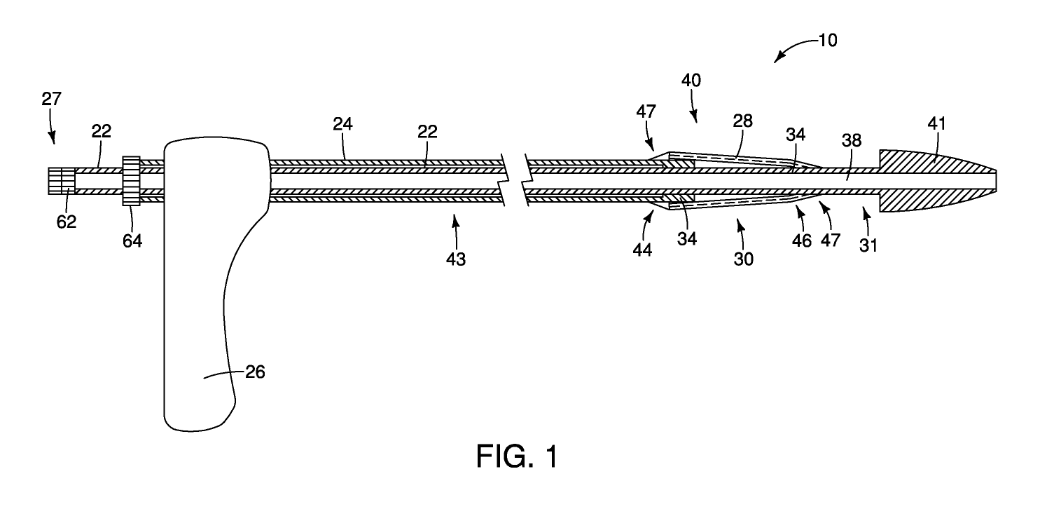

FIG. 1 illustrates a stent delivery system 10 for in accordance with embodiments of the present invention. The delivery system 10 may be provided as an over-the-wire configuration or a rapid exchange configuration. The stent delivery system 10 includes an inner shaft 22, an outer shaft 24 and a handle 26 at a proximal portion 27 of the system 10. A stent 28 is positionable on a stent region 30 of the inner shaft 22 and the outer shaft 24 at a distal portion 31 of the delivery system 10. One or more radiopaque markers 34 may be included on the delivery system 10 to indicate the position of the stent 28. The stent delivery system 10 may also include a guide wire (not shown) extendable through a port 38 of the inner shaft 22 through a distal tip 41 at the distal portion 31 of the delivery system 10.

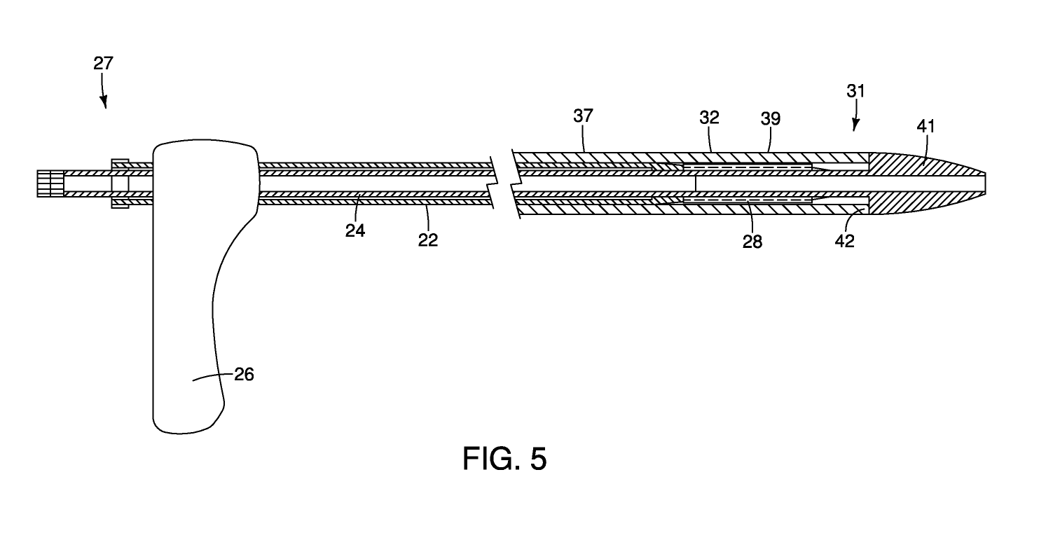

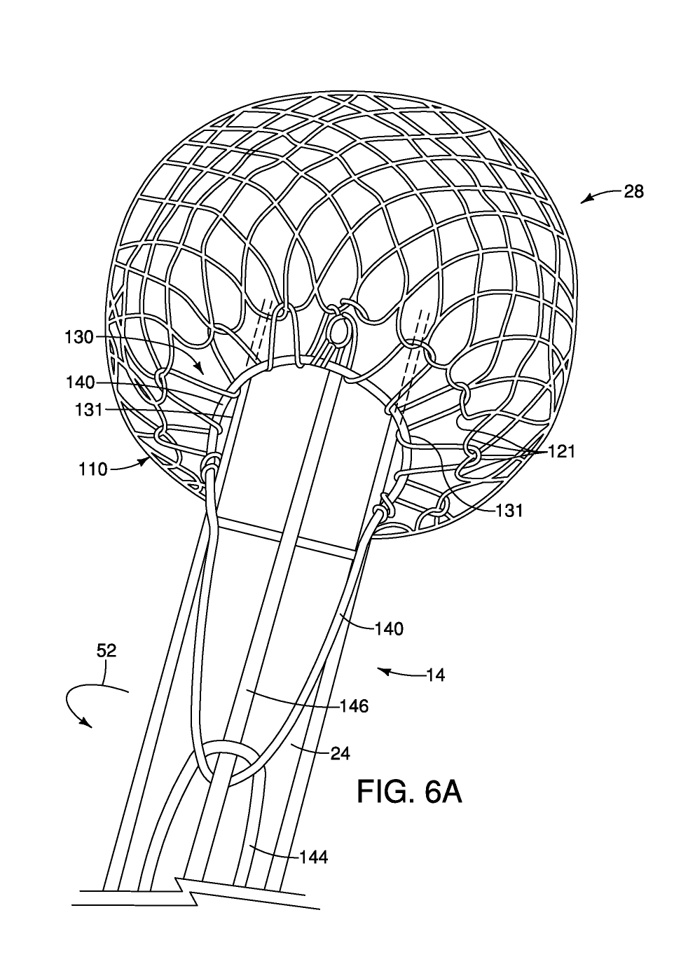

As shown in FIG. 1, the stent 28 is in a constrained configuration 40 collapsed against the inner shaft 22 and the outer shaft 24. The stent 28 is held in the constrained configuration 40 using a constraining system that does not require an outer sheath to constrain the stent. An embodiment of the constraining system is described in detail below with reference to FIGS. 6A-6B, that includes a proximal stent constraining member 44 and a distal stent constraining member 46 to torsionally constrain the stent 28 and hold the stent 28 collapsed against the inner and outer shafts 22, 24. The inner and outer shafts 22, 24 are in a first rotational position 43 when the stent 28 is in the constrained configuration 40. The proximal constraining member 44 is operably connected to the outer catheter 24 and the distal constraining member is operably connected to the inner catheter 22. The proximal and distal constraining members 44, 46 are in a first position 47 shown in FIG. 1. When present, an outer sheath 32 may provide some compressive force to the stent in addition to the proximal and distal constraining members 44, 46. (See FIG. 5.)

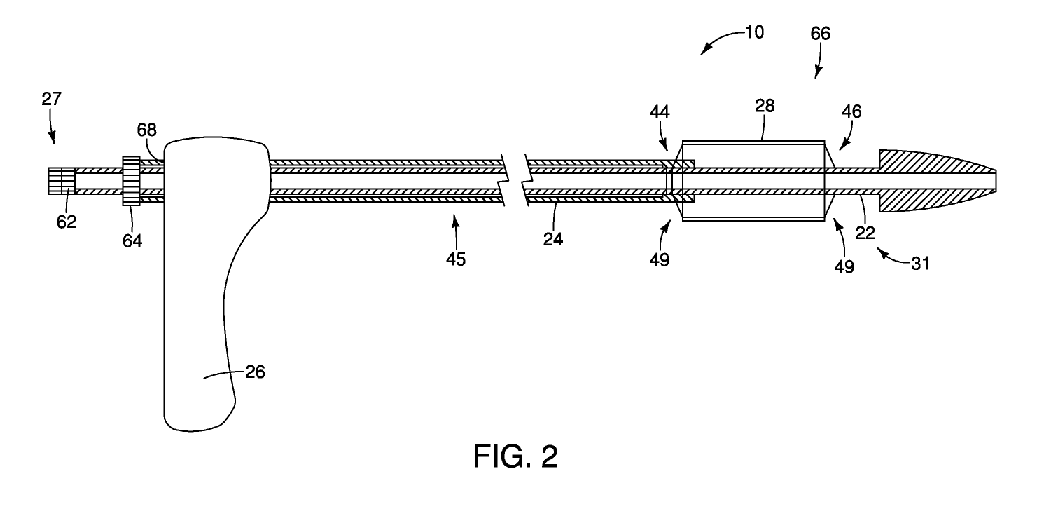

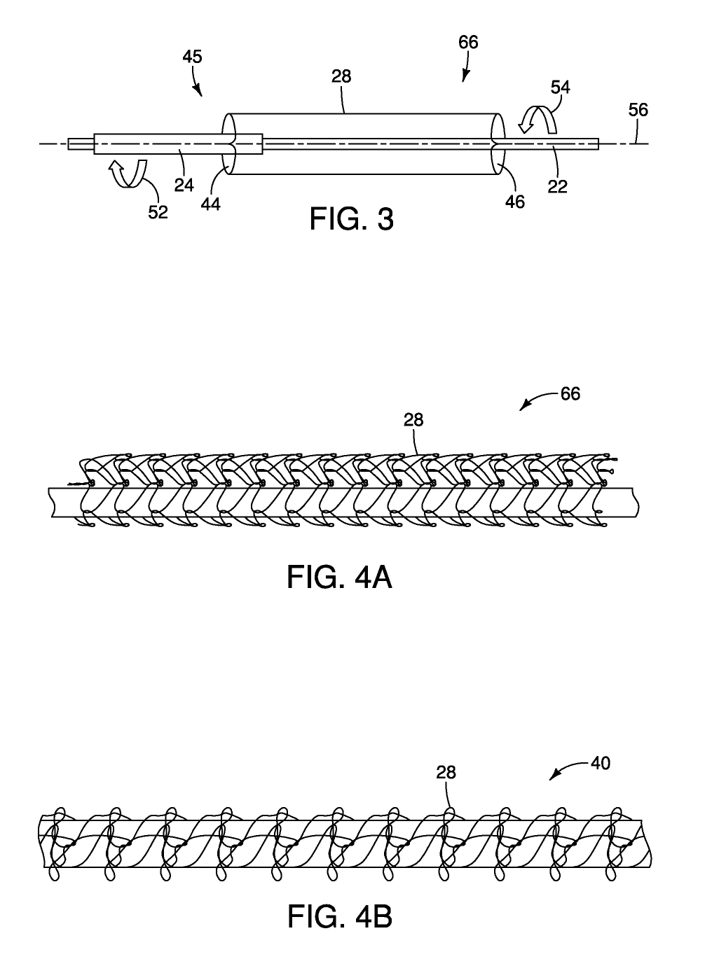

The stent 28 is shown in an expanded configuration 66 in FIG. 2 where the stent 28 is expanded away from the inner and outer shafts 22, 24. The proximal and distal constraining members 44, 46 are in a second position 49 and remain connected to the stent 28 but the torsional force on the stent 28 has been removed to allow the stent 28 to expand by rotation of the inner and outer shafts 22, 24 to a second rotational position 45. The inner shaft 22 and the outer shaft 24 are rotated in opposite directions relative to each other to the second rotational position 45 to release the tension on the proximal and distal constraining members 44, 46 to allow the stent 28 to expand to the expanded configuration 66. Expansion of the stent 28 is illustrated in FIG. 3 where the outer catheter 24 is rotated in a first direction 52 and the inner catheter 22 is rotated in a second direction 54 about a longitudinal axis 56 of the delivery system 10. In some embodiments, the inner shaft 22 or the outer shaft 24 may remain stationary and the other of the inner shaft 22 or the outer shaft 24 is rotated to expand the stent 28 to the expanded configuration 66. The torsional force on the stent 28 allows the stent 28 to collapse to the constrained configuration 40 and expand to the expanded configuration 66 without foreshortening the stent 28.

As shown in FIGS. 1 and 2, the delivery system 10 may include a first rotational member 62 operably connected to the inner shaft 22 and a second rotational member 64 connected to the outer shaft 24 to allow the operator to rotate the inner and/or outer shafts 22, 24 to torsionally constrain or unconstrain the stent 28. In other embodiments, the rotational movement of the inner and outer shafts 22, 24 may be controlled by a spring or other device. The handle 26 may also include a releasable lock 68 to lock the inner and outer shafts 22, 24 in position relative to each other, for example for navigation of the delivery device 10 through the bodily passageways to the treatment site. Once the stent 28 is in a treatment position, the lock 68 may be released so that the inner and/or outer shafts 22, 24 may be rotated to release the torsional force on the stent 28 to move the stent 28 from the constrained configuration 40 to the expanded configuration 66. Because the proximal and distal constraining members 44, 46 remain connected to the stent 28, the stent 28 may be re-constrained by rotation of the inner and/or outer shafts 22, 24 in the opposite direction to move the stent 28 to the constrained configuration 40. The stent 28 may be repositioned or withdrawn in the constrained configuration 40 and may be re-released to the expanded configuration 66 at a new site. The stent configurations may be changed multiple times within the patient for repositioning or removal until the proximal and distal constraining members 44, 46 are released from connection with the stent 28 as described below.

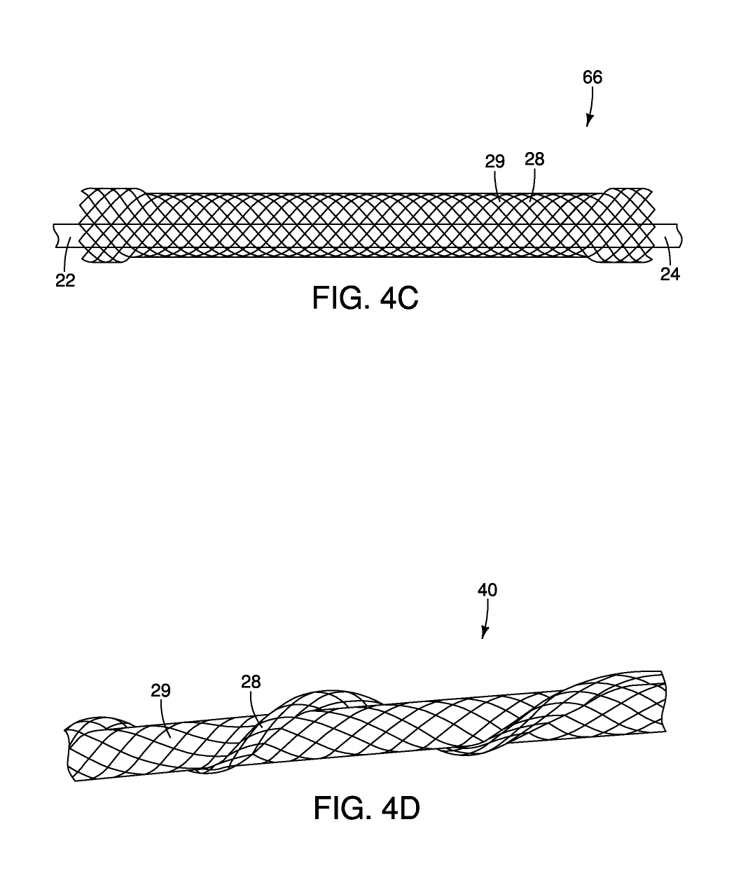

In some embodiments, the stent 28 may be a self-expanding stent. The stent 28 may be any kind of stent that has a tendency to radially collapse when a torsional force is applied to the ends of the stent. By way of non-limiting example, the stent 28 may be formed as a woven mesh formed from a metal or polymer or a laser cut pattern formed in a metal stent. The stent may also be formed from a bioabsorbable material. The stent may also be formed from a non-woven material. One example of a woven stent is the EVOLUTION® stent (Wilson-Cook Medical, Inc.) The stent 28 may also include a sleeve 29. (See FIGS. 4C and 4D.) FIGS. 4A-4D illustrate stents that may be used with the delivery system 10. FIGS. 4A and 4B illustrate an exemplary woven nitinol stent. FIG. 4A illustrates the stent 28 before torsion in the expanded configuration 66 and FIG. 4B illustrates the same stent 28 after torsion in the constrained configuration 40. FIGS. 4C and 4D illustrate a double helical woven nitinol stent having a sleeve in an expanded configuration 66 (FIG. 4C) and a constrained configuration 40 (FIG. 4D).

The stent delivery system 10 may optionally include an outer sheath 32 slidably positionable over a portion of the outer shaft 24 and the inner shaft 22 to cover the stent region 30 and the stent 28 as shown in FIG. 5. The optional outer sheath 32 is shown extended distally over the stent 28 and abutting the distal tip 41 of the inner shaft 22 forming a smooth outer surface 42 of the delivery system 10. The outer sheath 32 may be operably connected to the handle 26 and may be proximally withdrawn to expose the stent 28 in the constrained configuration 40 (see FIG. 1). The outer sheath 32 may be provided to facilitate a smoother delivery of the system 10 through a bodily lumen of the patient.

An exemplary embodiment of the proximal constraining member 44 is illustrated in FIG. 6A. The proximal constraining member 44 is connected to the outer shaft 24. The distal constraining member 46 is similarly configured to the proximal constraining member 44 and is connected to the inner shaft 22 rather than the outer shaft 24 and is shown in FIG. 6B

The proximal constraining member 44 may include an outer filament 130 and an inner filament 140. The outer filament 130 may be interwoven through one or more peaks 121 at an end portion 110 of the stent 28. The inner filament 140 engages with the outer filament 130 to pull the outer filament 130 taught and to reduce the diameter of the stent end portion 110 and close the end portion 110 against the outer catheter 24 as shown in FIG. 6A. Rotation of the outer catheter 24 further collapses the stent 28 against the outer catheter 24, for example by rotating the outer catheter 24 in the first direction 52. (See FIGS. 4B, 4D, and 4F showing the collapsed stent 28 in the constrained configuration 40.) The proximal constraining member 44 may further include a retaining loop 144 that may be attached to the outer shaft 24. The proximal constraining member 44 may also include a proximal retaining wire 146 that is configured to cooperate with the inner filament 140 and the retaining loop 144 to releasably lock the inner filament 140 to the retaining loop 144 to allow selective expansion and contraction of the stent 28 when the inner and/or outer shafts 22, 24 are rotated to torsionally constrain and unconstrain the stent 28.

An exemplary cooperative configuration of the proximal constraining member 44 is shown in FIG. 6A where a portion of the inner filament 140 and the retaining loop 144 are overlapping and the proximal retaining wire 146 extends through the overlapping loops of the inner filament 140 and the retaining loop 146 to releasably hold the two loops 140, 146 together. The proximal retaining wire 146 shown in FIG. 6 may be frictionally engaged with a portion of the outer shaft 24 to hold the proximal retaining wire 146 in position until the stent 28 is in the proper position for release as discussed above. The proximal retaining wire 146 may be proximally withdrawn to release the proximal constraining member 44 and to completely release the stent 28 from connection to the outer shaft 24.

As shown in FIG. 6B, the distal constraining member 46 is connected to the inner shaft 22 with the stent 28 in the expanded configuration 66. The inner and outer filaments 130, 140 are relaxed so the stent 28 is expanded, but still connected to the inner shaft 22. The distal constraining member 46 may further include a distal loop 176 that cooperates with the inner filament 140 and a distal retaining wire 186 to connect the stent 28 to the inner catheter 22, similar to the configuration described above for the proximal constraining member 44.

The proximal and distal retaining wires 146, 186 may be connected to the handle 26 for proximal withdrawal of the proximal and distal retaining wires 146, 186 from the loops of the outer filaments 140, the proximal and distal loops 144, 176 to completely release the stent 28 from the delivery system 10. The withdrawal of the proximal and distal retaining wires 146, 176 may be simultaneous or sequential. Because the stent 28 has been expanded and positioned in the proper position within the lumen of the patient, the timing of the release of the retaining wires 146, 186 is not critical for the positioning of the stent 28. As will be understood by one skilled in the art, the proximal constraining member 44 may be connected to the inner catheter 22 and the distal constraining member 46 may be connected to the outer catheter 24. In embodiments provided without the outer sheath 32, the peaks 121 of the stent 28 are collapsed closely against the inner and outer shafts 22, 24 for delivery to the patient site.

While the proximal and distal restraining members 44, 46 have been described with reference to connection to the end portions 110 of the stent 28, it is also possible to provide proximal and distal constraining members 44, 46 that are connected to other portions of the stent 28 and still provide a constrained configuration 40 for the stent 28. For example, the proximal constraining member may be connected to a mid proximal portion or mid-point of the stent and the distal constraining member may be connected to the distal end portion of the stent. Similarly, the proximal constraining member may be connected to the proximal end portion of the stent and the distal constraining member may be connected to the midpoint of mid distal portion of the stent or both the proximal and distal constraining members may be connected to other than the proximal and distal end portions of the stent. In some embodiments, the proximal or the distal constraining members or both proximal and distal constraining members may be connected to the stent at a plurality of positions on the stent.

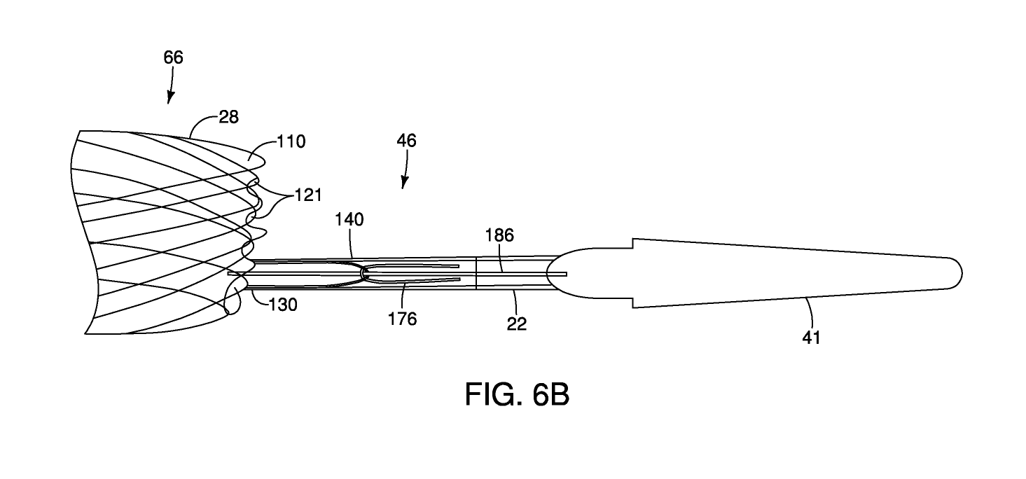

In some embodiments, the stent delivery system 10 may be provided with proximal and distal constraining members 44, 46 having the outer filament 140 woven through the peaks 121 at the end portion 110 of the stent 28 without the inner filament. The outer filament 140 is shown woven though the peaks 121 in FIG. 7. The outer filament 140 may be connected to a proximal or distal loop 144, 176 and cooperatively connected to the inner or outer shaft 22, 24 by the retaining wire 146, 176 as described above with reference to FIGS. 6A and 6B.

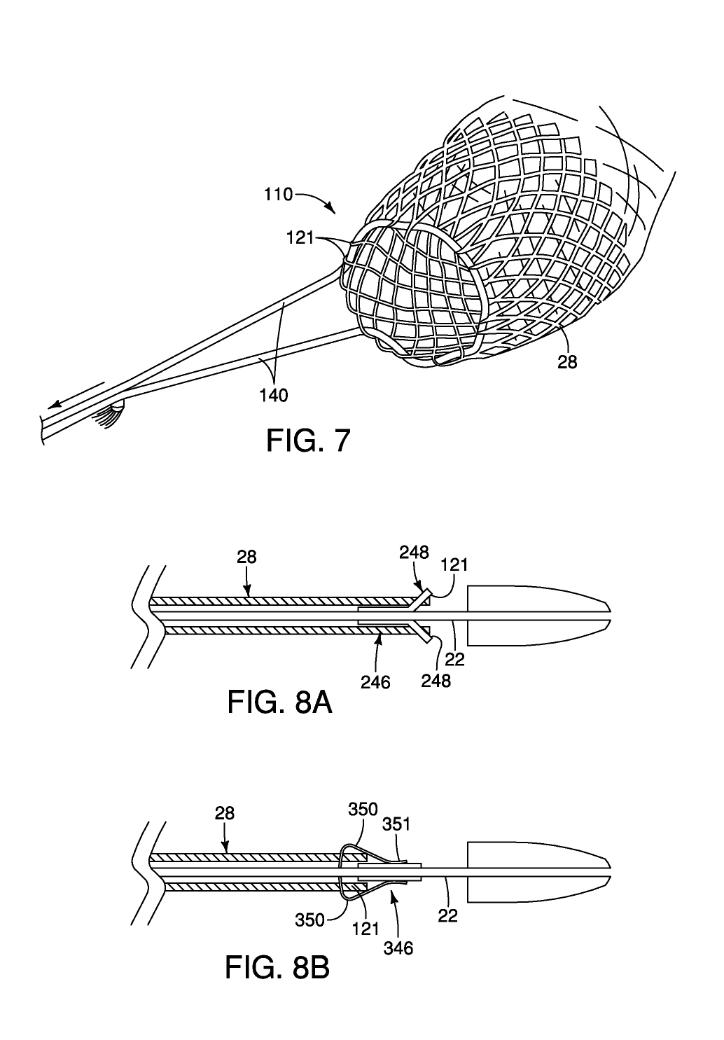

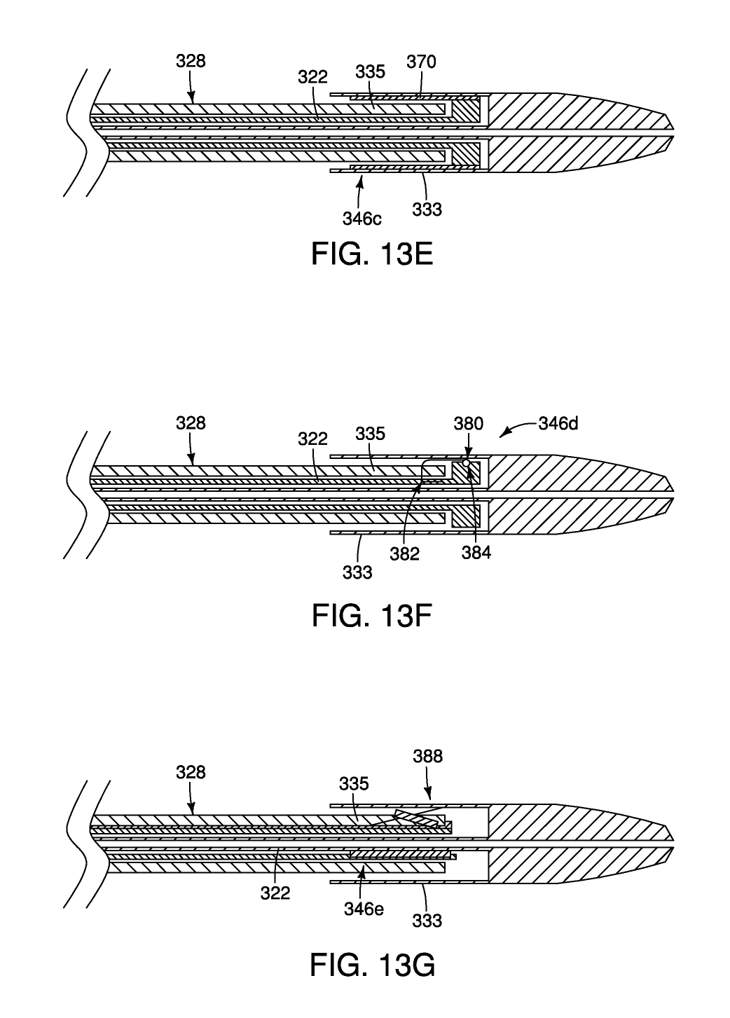

Additional configurations for the proximal and distal constraining members are also possible. By way of non-limiting example, additional configurations for alternative embodiments of the constraining members are shown in FIGS. 8A, 8B and 13A-13G. A similar proximal constraining member is also provided, but not shown. The proximal and distal constraining members may be the same or different. FIGS. 8A and 8B illustrate alternative embodiments of a distal constraining member 246, 346. The distal constraining member 246 shown in FIG. 8A includes one or more hooks 248 that may hook onto peaks 121 of the stent 28 to constrain the stent 28 on the inner shaft 22. A plurality of hooks 248 may be provided on the inner shaft 22 and spaced apart to evenly hold the stent 28 in position. For example, 4 hooks may be provided and spaced apart by 90°. The hooks 248 may also be skived from the inner shaft 22. Other combinations of numbers of hooks and spacing of the hooks may also be provided, including uneven spacing and uneven numbers of hooks. One or more hooks 248 may be provided with a retaining wire 288 (not shown) extending through the hook 248 and the stent peak 121 to releasably lock the stent 28 to the delivery system 10, for example, similar to the embodiment described above with reference to FIGS. 6A-6B.

The distal constraining member 246 may also include a loop (not shown) similar to the loop 176 described in FIGS. 6A and 6B above that is woven between the peaks 121 and the hook 248 connects to the loop 176 to constrain the stent 28. The hook 248 may be released from the stent peak 121 or the loop 176 by rotating the proximal and/or distal constraining members 244, 246 and reducing the torsional force on the stent 28 so that the stent 28 expands and releases the hook 248. The hook 248 may also be released by withdrawing the retaining wire 288 and releasing the lock between the peak 121 and the hook 248, for example. The stent 28 may be expanded and constrained a plurality of times prior to release of the retaining wire 288 similar to the embodiments described above.

FIG. 8B illustrates the distal constraining member 346 that includes one or more grasping members 350 that grasp a portion of a stent 28 to hold the stent on the inner shaft 22. The grasping members 350 may be provided on the inner shaft 22 and spaced apart to hold the stent 28 in position similar to the arrangements described above for the hooks 228. One or more grasping members 350 may be provided with a retaining wire 388 (not shown) extending through the grasping member 350 and the stent 28 to releasably lock the stent 28 to the delivery system 10 similar to the embodiments described above. The distal constraining member 346 may also include a loop 382 (not shown) similar to the loop 176 described above that is woven between the peaks 121 of the stent 28 and the grasping member 350 connects to the loop 382 to hold the stent 28 while the inner and/or outer shafts 22, 24 are rotated to provide torsional force on the stent 28 to hold the stent in the constrained configuration 40. The grasping member 350 may be released from the stent 28 or the loop 382 by opening the grasping member 350 away from the stent 28 after the torsional force has been released, for example by pressing on a distal portion 351 of the grasping member 350 to flex the grasping member 350 open. The stent 28 may be expanded and constrained a plurality of times prior to release of the retaining wire 388 similar to the embodiments described above.

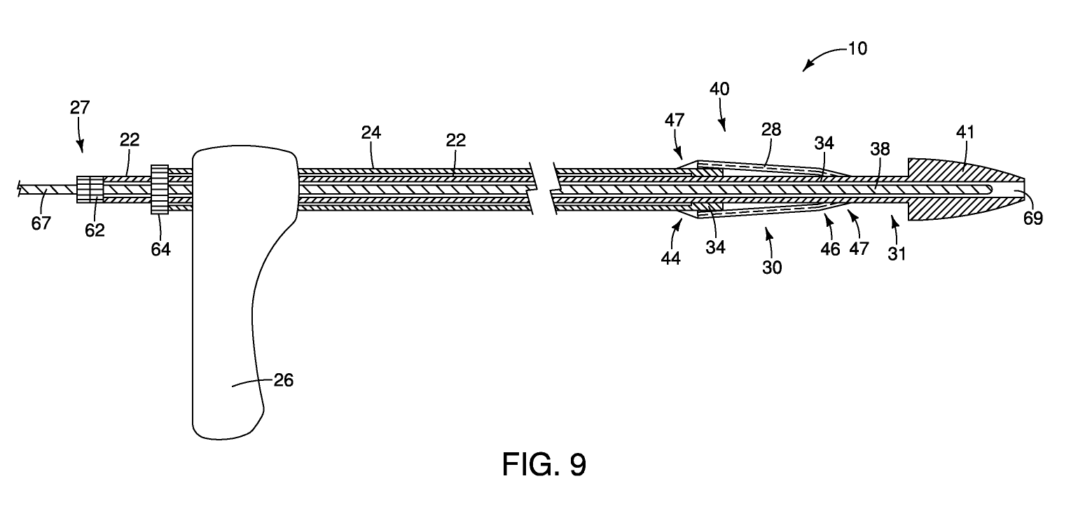

In some embodiments, a stiffening member 67 may be removably provided in a lumen 69 of the inner shaft 22 as shown in FIG. 9. The stiffening member may be provided as a mandril, catheter, rod and the like that is removably insertable into the lumen 69. The stiffening member 67 may be provided to help increase the rigidity of the inner catheter 22 against the inward torsional force of the stent 28 when the stent 28 is in the constrained configuration 40. In some embodiments, the inner shaft 22 may be provided in a soft material to facilitate passage through the body lumen. In the event that the materials are sufficiently soft, the inner catheter 22 may collapse or deform in response to the torsional force of the stent 28 provided by the first and second constraining members 44, 46 torsionally constraining the stent 28 against the inner shaft 22 and outer shaft 24. The stiffening member 67 may be made from any material having suitable stiffness to provide support for the inner shaft 22 with the stent 28 torsionally constrained on the inner shaft 22 and the outer shaft 24. Exemplary materials for forming the shaft include, but are not limited to, metal alloys such as stainless steel, tantalum or its alloys, tungsten, platinum, gold, copper, palladium, rhodium, or a superelastic alloys, such as nitinol or polymers that can be provided with sufficient shore hardness, such as Pebax, Peek, polyimide, liquid crystal polymers (LCP) such as Vectran, polyethylene, polyethylene terephthalate and Nylon. As shown in FIG. 5, the outer sheath 32 may be provided for delivery of the stent to the area of the treatment site. The outer sheath 32 compresses the stent 28 against the inner shaft 22 for delivery of the device 10 to the treatment site with the stiffening member 67 removed and the stent 28 in the constrained configuration 40. The stiffening member 67 may be inserted into the lumen 69 when the stent 28 is near the proper position for implantation into the patient and the outer sheath 32 is over the stent 28 as shown in FIG. 5. The outer sheath 32 may be withdrawn and the stent 28 remains constrained on the inner shaft 22 by the proximal and distal constraining members 44, 46 providing torsional force. The stiffening member 67 supports the inner shaft 22 against the compressive torsional force exerted by the proximal and distal constraining members 44, 46.

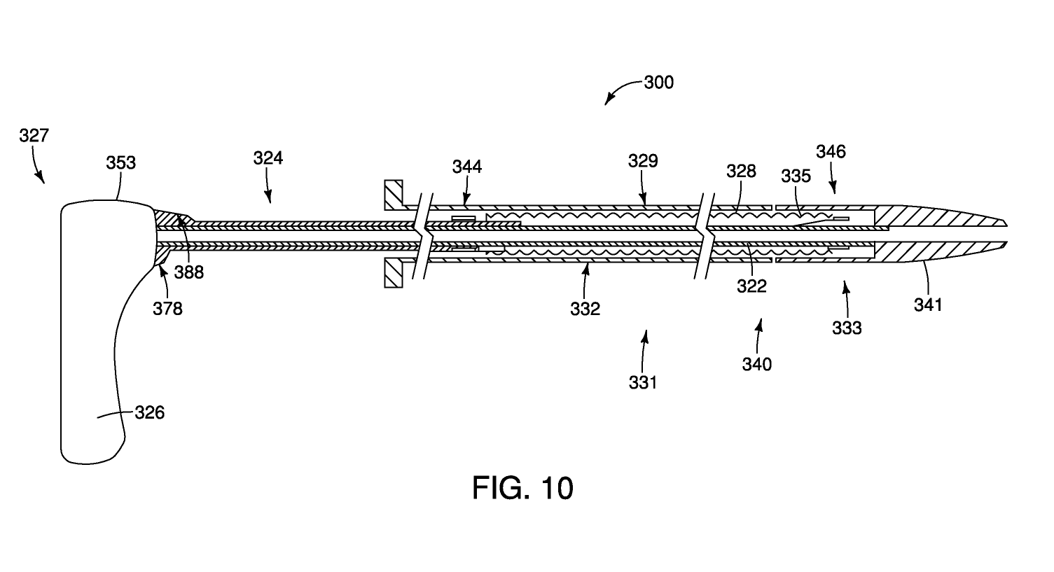

FIGS. 10-12 illustrate a stent delivery system 300 in accordance with another embodiment of the present invention. The stent delivery system 300 includes an inner shaft 322, an outer shaft 324 and a handle 326 at a proximal portion 327 of the system 300. A stent 328 is positionable on the inner shaft 322 and outer shaft 324 at a distal portion 331 of the delivery system 300. The stent delivery system 300 may optionally include an outer proximal sheath 332 and an outer distal sheath 333 slidably positionable over a portion of the inner shaft 322 and the outer shaft 324 to cover the stent 328. The proximal outer sheath 332 may be proximally withdrawn and the distal outer sheath 333 may be separately and distally withdrawn to expose the stent 328. A guide wire (not shown) may be extendable through the inner shaft 322 to a distal tip 341 at the distal portion 331 of the stent delivery system 300. An optional stiffening member may be insertable into the inner shaft 322 similar to the stiffening member 67 described above with reference to FIG. 9.

As shown in FIG. 10, the stent 328 is in a constrained configuration 340 collapsed against the inner shaft 322 and the outer shaft 324. The optional proximal outer sheath 332 is shown extended over a proximal portion 329 of the stent 328. The distal outer sheath 333 is shown extended over a distal portion 335 of the stent 328. The stent 328 is held in the constrained configuration by a proximal constraining member 344 and a distal constraining member 346 that provide a torsional force when the inner shaft 322 and/or outer shaft 324 are rotated to hold the stent 328 in the constrained configuration 340. When present, the outer sheath 322 may provide some compressive force to the stent 328 in addition to the proximal and distal constraining members 344, 346. Alternatively or additionally, the distal portion 335 of the stent 328 may constrained by the distal outer sheath 333. The proximal and distal constraining members 344, 346 are operably connected to the handle 326. A lock 353 may be provided to releasably lock the inner and outer shafts 322, 324 in position relative to each other.

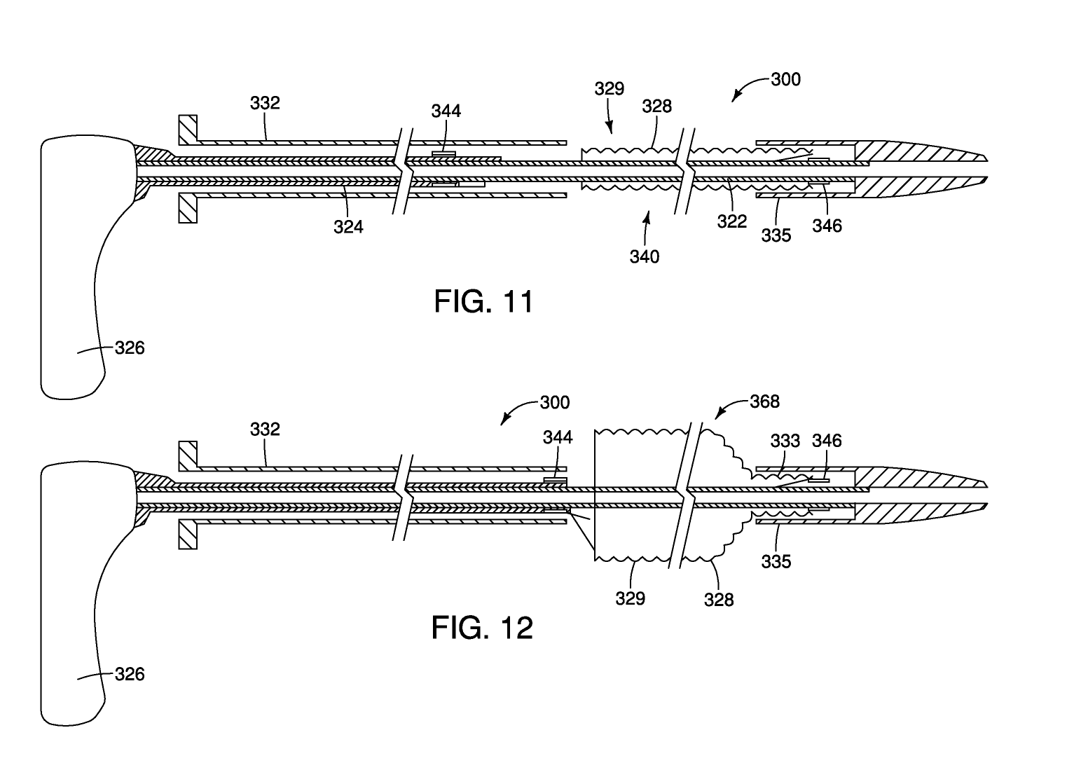

As shown in FIG. 11, the proximal outer sheath 332 has been proximally withdrawn exposing the proximal portion 329 of the stent 328. The stent 328 is held in the constrained configuration 340 by the proximal constraining member 344 and the distal constraining member 346 with the inner and outer shafts 322, 324 rotated to provide torsional force. The distal outer sheath 333 is positioned over the distal portion 335 of the stent 328.

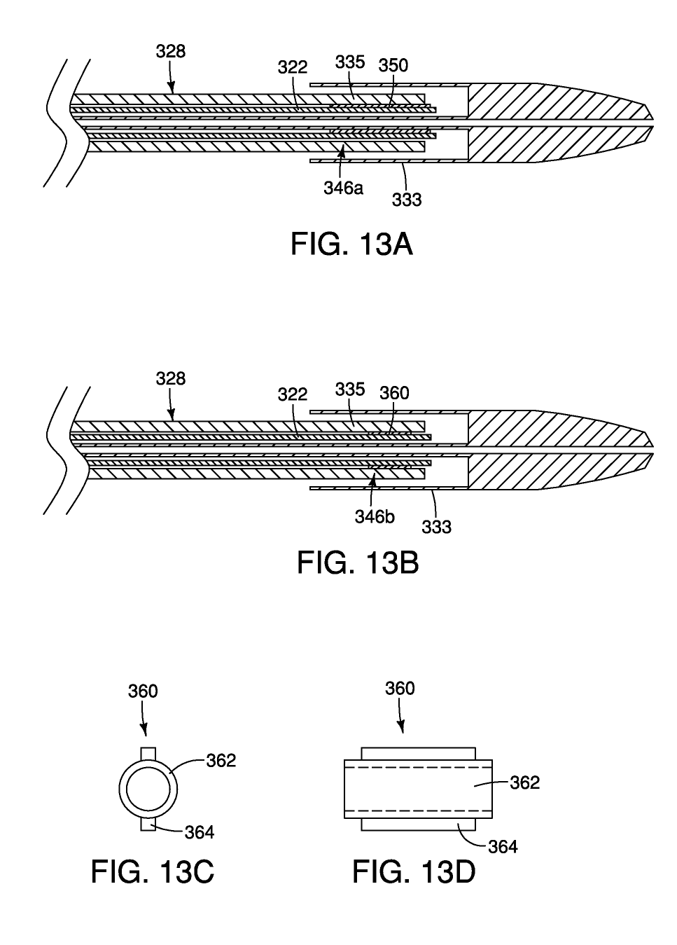

FIG. 12 illustrates the stent 328 with the proximal portion 329 of the stent 328 in an expanded configuration 368. The distal portion 335 of the stent 328 is covered by the distal outer sheath 333 and remains collapsed against the inner shaft 322 by the outer sheath 333. The inner shaft 322 and/or the outer shaft 324 have been rotated to release the torsional force on the stent 328. As shown in FIG. 12, the stent 328 may be expanded at the proximal portion 329 for proper placement of the proximal portion 329 within the patient's lumen and then the distal portion 335 of the stent 328 may be expanded by the distal withdrawal of the outer distal sheath 333 to allow the distal portion 335 to expand. Both the proximal and distal constraining members 344, 346 remain connected to the stent 328 in the expanded configuration 368 to allow the stent 328 to be repositioned between the expanded and constrained configurations 340, 368 until the stent 328 is optimally positioned within the patient's lumen. Similar to the delivery systems described above, the stent 328 may be completely released from the proximal and distal constraining members 344, 346 by removal of restraining members 378, 388 that are part of the proximal and distal constraining members 344, 346.

As will be understood by one skilled in the art, the stent delivery system 300 may also be used to release the distal portion 335 of the stent 328 first to expand and position the distal portion 335 of the stent 328. The inner shaft 322 and/or the outer shaft 324 are rotated to release the torsional force on the stent 328 and the proximal portion 329 remains collapsed against the outer shaft 324 by the outer sheath 332. The stent 328 may be recollapsed by providing torsional force by rotating the inner shaft and/or outer shaft as described above until the proximal and distal constraining members 344, 346 are completely released, for example, by removal of the restraining members 378, 388

FIGS. 13A-13G illustrate alternative embodiments of a distal constraining member 346a-346e that operates similarly to the distal constraining member 346 described above to move the stent 328 between the constrained configuration 340 and the expanded configuration 368. A similar proximal constraining member may also be provided for each of the embodiments of the distal constraining members 346a-346e, but not shown, and may be connected to the outer shaft and cooperatively work with the distal constraining member 346a-346e. Only reference to the distal portion will be made. The distal constraining member 346a shown in FIG. 13A includes a high friction surface 350 on the inner shaft 322. By way of non-limiting example, the high friction surface 350 may be a material with a high coefficient of friction or a coating of a releasable adhesive. The high friction surface 350 is positioned between the inner shaft 322 and the distal outer sheath 333 so that the distal portion 335 of the stent 328 is releasably locked to the inner shaft 322. With the distal outer sheath 333 positioned over the distal portion 335 of the stent 328, the stent 328 may be moved between the expanded configuration 368 and the constrained configuration 340 as shown in FIGS. 11 and 12 by rotating the inner shaft 322 and/or the outer shaft 324 to provide torsional force on the stent 328. The stent 328 may be released from the inner shaft 322 by distally withdrawing the distal outer sheath 333 so that the distal portion 335 of the stent 328 is exposed and expanded. A distal retaining member 388 may be included and threaded though the distal portion 335 of the stent 328 to reliably lock the distal portion 335 of the stent 328 to the inner shaft 322.

An alternative embodiment of the distal constraining member 346b shown in FIGS. 13B-13D includes a mechanical fixing member 360 on the inner shaft 322. By way of non-limiting example, the mechanical fixing member 360 may include a tube 362 with at least one tab 364. Wires of the stent 328 at the distal portion 335 are releasably held in position over the tabs 364 and the distal outer sheath 333 is shown positioned over the distal portion 335 of the stent 328. A cross section of the mechanical fixing member 360 is shown in FIG. 13B and a longitudinal section of the mechanical fixing member 360 is shown in FIG. 13C. Other shapes and patterns of tabs 364 may also be used. The tube 362 may be crimped or glued to the inner shaft 322 or the tabs 364 may be connected to the inner shaft 322 by other means known to one skilled in the art. The distal portion 335 of the stent 328 may automatically disengage from the tabs 364 when the distal outer sheath 333 is distally withdrawn from the stent 328 to release the stent to the expanded configuration 369.

An alternative embodiment of the distal constraining member 346c shown in FIG. 13E and includes a sleeve member 370 on the inner shaft 322. The sleeve member 370 may be made of a high friction material similar to the high friction surface 350. The sleeve member 370 is positioned between the stent 328 and the distal outer sheath 333 so that the distal portion 335 of the stent 328 is releasably locked to the inner shaft 322. The stent 328 may be released from the inner shaft 322 by distally withdrawing the distal outer sheath 333 and the sleeve member 370 so that the distal end 335 of the stent 328 is exposed and the stent 328 expanded to the expanded configuration 369.

An alternative embodiment of the distal constraining member 346d shown in FIG. 13F. The distal constraining member 346d includes at least one bead 380, each bead 380 connected to a line 382. The line 382 is connected to the inner shaft 322 and extents through a loop of the stent 328 at the distal portion 335. The inner shaft 322 includes a receptacle 384 sized and shaped to receive the bead 380 and allow the outer distal sheath 333 to slide over the distal portion 335 of the stent 328. The bead 380 positioned in the receptacle 380 and the distal outer sheath 333 over the bead 380 to releasably hold the stent 328 in the constrained configuration 340. The stent 328 may be released from the inner shaft 322 by distally withdrawing the distal outer sheath 333 and releasing the bead 380 from the receptacle 380 so that the stent 328 is released and the bead 380 connected to the line 382 is withdrawn through the loop of the stent 328 and remains with the inner shaft 322 with the stent 328 in the expanded configuration 340.

An alternative embodiment of the distal constraining member 346e shown in FIG. 13G and includes a retaining wire 388 extending through a loop of the stent 328 in the distal portion 335. The distal outer sheath 333 is positioned over the retaining wire 388 through the stent loop so that the distal portion 335 of the stent 328 is releasably locked to the inner shaft 322. The stent 328 may be released from the inner shaft 322 by distally withdrawing the distal outer sheath 333 and proximally withdrawing the retaining wire 388. The distal end 335 of the stent 328 is exposed and released and the stent 628 expands to the expanded configuration 369.

The materials used to manufacture the components of the stent delivery systems described herein may be any materials known to one skilled in the art that are suitable for use in patients. By way of non-limiting example, the shafts and sheaths may be formed from polytetrafluorothylene (PTFE) particularly when a low friction outer sheath is desirable. Nylon and HDPE may also be used for clarity. Additional possible materials include, but are not limited to the following, polyethylene ether ketone (PEEK), fluorinated ethylene propylene (FEP), perfluoroalkoxy polymer resin (PFA), polyamide, polyurethane, high density or low density polyethylene, and nylon including multi-layer or single layer structures and the like and may also include reinforcement wires, braid wires, coils, coil springs and or filaments. The stent may be formed from but is not limited to the following materials: Nickel titanium alloys, for example, nitinol, stainless steel, cobalt alloys and titanium alloys. The loops of the constraining members may be made from common suture material as known in the art, for example polyester suture such as 4-0 Tevdek®, nylon, silk, polypropylene, ultra high molecular weight polyethylene (UHMPE) and the like. The sutures may be monofilament, braided, twisted or multifilament. The loops and the retaining wires may also be made from a metallic alloy such as stainless steel or nickel titanium. In some embodiments, the stent, the loops and/or the retaining wires may be made from bioderadable materials. A number of bioabsorbable homopolymers, copolymers, or blends of bioabsorbable polymers are known in the medical arts. These include, but are not necessarily limited to, polyesters including poly-alpha hydroxy and poly-beta hydroxy polyesters, polycaprolactone, polyglycolic acid, polyether-esters, poly(p-dioxanone), polyoxaesters; polyphosphazenes; polyanhydrides; polycarbonates including polytrimethylene carbonate and poly(iminocarbonate); polyesteramides; polyurethanes; polyisocyantes; polyphosphazines; polyethers including polyglycols polyorthoesters; expoxy polymers including polyethylene oxide; polysaccharides including cellulose, chitin, dextran, starch, hydroxyethyl starch, polygluconate, hyaluronic acid; polyamides including polyamino acids, polyester-amides, polyglutamic acid, poly-lysine, gelatin, fibrin, fibrinogen, casein, collagen.

Other suitable biocompatible materials may also be used for any of the components described herein.

Operation of the stent delivery systems of the present invention is described with reference to the stent delivery system 10 by way of non-limiting example. Alternative methods of operating the system may also be used. The stent delivery system 10 may be provided in a sterile packaging. The stent 28 may be provided in the expanded configuration 66 or constrained configuration 40 within the packaging. For example, some stent materials may weaken or otherwise deform when stored in a constrained configuration 40 with the torsional force being exerted on the stent during shipping and storage. In some embodiments provided with an outer sheath 32, the outer sheath 32 may be provided to hold the stent 28 in position on the inner and outer shafts 22, 24 without being rotated to provide the torsional force. For example, the system 10 may be provided with the inner shaft 22 and outer shaft 24 positioned in relation to each other such that no torsional force is provided to the stent 28 and the outer sheath 32 is positioned over the stent 28 to hold the stent on the inner shaft 22 and outer shaft 24. Prior to insertion of the distal portion 31 of the system 10 into the patient, the operator may rotate the inner shaft 22 and/or outer shaft 24 to place torsional force on the stent 28 using the proximal and distal constraining members 44, 46 to constrain the stent 28. The stent 28 may be provided in the expanded configuration 66 in the absence of a sheath as well and be moved to the constrained configuration 40 by rotation of the inner shaft 22 and/or outer shaft 24 to provide torsional force to the stent 28 prior to insertion of the stent into the patient.

Minimal fluoroscopy may be used for placement of the stent 28 within the patient's lumen because of the lack of foreshortening when the stent is released from the delivery system. The rotational release of the torsional force on the stent 28 means that the midpoint of the stent 28 in the constrained configuration 40 on the inner shaft 22 and outer shaft 24 is the midpoint when the stent 28 is released also so that the stent 28 can be precisely positioned based on the known midpoint of the stent 28. Fluoroscopy is not required during placement of the stent 28 once the placement position has been determined. The stricture length within the patient's lumen at the treatment site is measured using fluoroscopy. Then the stent 28 may be placed at the proper position within the lumen using an endoscope alone.

The outer sheath 32 may include two different sets of distance measurement markings 37, 39, one to be used when the outer sheath 32 is covering the stent 28 and one set to be used when the outer sheath 32 has been withdrawn (See FIG. 5). The markings 37, 39 may be of different colors, for example, to easily identify the two measurements. The operator measures the distance from the incisor teeth to the midpoint of the stricture. The stent delivery system 10 is inserted into the patient using the first set of sheath markings 37 to place the constrained stent 28 in the stricture by measuring the distance relative to the incisor teeth. The sheath 32 is withdrawn proximally and locked to the handle 26 to expose the stent 28. The second set of markings 39 is used once the sheath 32 is withdrawn to measure the distance between the stricture and the incisor teeth to ensure that the stent 28 is still in the correct position relative to the stricture. Because the outer sheath 32 is not used to deploy the stent 28, the markings 37, 39 can be placed clearly on the outside of the sheath and the outer sheath can be locked to the handle 26 and held steady relative to the patient's incisor teeth to increase the accuracy of the stent placement.

The endoscope is positioned within the lumen so the operator can view the proximal side of the stricture. A guide wire is inserted through the stricture and the endoscope is removed. The proper length stent 28 is selected based on the stricture measurement. The operator inserts the distal portion 31 of the stent delivery system into the patient's lumen with the stent 28 in the constrained configuration 40 on the inner shaft 22. The guidewire may be inserted first to navigate a tortuous pathway to the treatment site and the system 10 is delivered over the guidewire to the treatment site. The endoscope may then be placed into the patient's lumen adjacent and parallel to the system 10. Alternatively, the stent delivery system 10 may be inserted into the patient's lumen through the working channel of an endoscope, depending on the size and location of the lumen.

A viewing port of the endoscope is used to identify the proximal end of the stricture at the treatment site. The stent region 30 is positioned within the lumen at the treatment point. For embodiments having a softer inner shaft 22, the stiffening member 67 is inserted through the lumen 69 of the inner shaft 22 to provide support for the longitudinally tensioned stent. The outer sheath 32, if present, is proximally withdrawn and the stent 28 in the constrained configuration 40 is exposed within the patient's lumen. The constrained stent 28 may be moved within the lumen to correctly position the stent 28 at the treatment site. The stent 28 is moved to the expanded configuration 66 by rotation of the inner shaft 22 relative to the outer shaft 24 so that the proximal and distal constraining members 44, 46 are moved to the second position 49 releasing the torsional force on the stent 28. The position of the expanded stent 28 is monitored using the endoscope. The stent 28 may be returned to the constrained configuration 40 by the operator rotating the inner shaft 22 relative to the outer shaft 24 and returning the proximal and distal constraining members 44, 46 to the first position 47 to exert torsional force on the stent 28 against the inner shaft 22 and outer shaft 24, for example if the stent 28 is incorrectly positioned. The stent 28 may be moved from the constrained configuration 40 to the expanded configuration 66 as many times as needed.

Once the proper position for the stent 28 is achieved within the patient's lumen, the proximal and distal retaining wires 78, 88 may be proximally withdrawn from the stent 28 to completely release the stent 28 from the proximal and distal constraining members 44, 46. The delivery system 10 is withdrawn proximally from the patient and the endoscope removed.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

1. A stent delivery system comprising:
an inner elongate shaft including a proximal portion, a distal portion, a lumen extending at least partially therethrough;
an outer elongate shaft including a proximal portion, a distal portion, the outer elongate shaft coaxially positioned over at least a portion of the inner elongate shaft; the inner elongate shaft being rotatably positionable relative to the outer elongate shaft about a longitudinal axis from a first rotational position to a second rotational position;
a stent having a proximal portion and a distal portion, the stent positioned on at least a portion of the inner elongate shaft, the stent having a constrained configuration and an expanded configuration;
a proximal constraining member connected to a proximal portion of the stent;
a distal constraining member connected to a distal portion of the stent; the proximal constraining member and the distal constraining member configured to cooperatively apply a torsional force to at least a portion of the stent in the constrained configuration with the inner and outer elongate shafts in the first rotational position.

2. The stent delivery system of claim 1, wherein the inner and the outer elongate shafts are both rotatably positionable.

3. The stent delivery system of claim 1, wherein the proximal constraining member comprises a proximal retaining wire and the distal constraining member comprises a distal retaining wire to releasably lock the stent to the proximal and distal constraining members.

4. The stent delivery device of claim 3, wherein the proximal and distal constraining members each comprise a loop releasably connected to the stent and one retaining wire.

5. The stent delivery system of claim 3, wherein the proximal and distal retaining wires are removable from connection with the stent to completely release the stent from the proximal and distal constraining members.

6. The stent delivery system of claim 1, wherein the stent is repeatedly movable between the constrained configuration and the expanded configuration.

7. The stent delivery system of claim 1, wherein the proximal and distal constraining members each comprise a first filament interwoven through a plurality of peaks of a stent end, each first filament configured to be pulled to collapse each stent end radially inward.

8. The stent delivery system of claim 7, wherein the proximal and distal constraining members each comprise a second filament interwoven through loops of the first filament.

9. The stent delivery system of claim 1 wherein the proximal and distal constraining members each comprise at least one hook.

10. The stent delivery system of claim 9, wherein the proximal and distal constraining members each comprise a retaining wire.

11. The stent delivery system of claim 1, further comprising a stiffening member removably positionable in the lumen to support the inner elongate shaft against the torsional force applied to the stent.

12. A stent delivery system comprising:
an inner elongate shaft including a proximal portion, a distal portion, a lumen extending at least partially therethrough;
an outer elongate shaft including a proximal portion, a distal portion, the outer elongate shaft coaxially positioned over at least a portion of the inner elongate shaft; the inner elongate shaft being rotatably positionable relative to the outer elongate shaft about a longitudinal axis from a first rotational position to a second rotational position;
a stent having a proximal portion and a distal portion, the stent positioned on at least a portion of the inner elongate shaft, the stent having a constrained configuration and an expanded configuration, the distal portion of the stent operably connected to the inner elongate shaft and the proximal portion of the stent operably connected to the outer elongate shaft;
the inner elongate member rotatably positionable relative to the outer elongate member to cooperatively apply a torsional force to at least a portion of the stent in the constrained configuration.

13. The stent delivery system of claim 12, further comprising a proximal constraining member connected to a proximal portion of the stent and a distal constraining member connected to a distal portion of the stent.

14. A method of implanting a stent in a patient's lumen, the method comprising:
inserting a distal portion of a stent delivery system into the lumen of a patient, the stent delivery system comprising:
an inner shaft including a proximal portion, a distal portion, a lumen extending at least partially therethrough,
an outer shaft including a proximal portion, a distal portion, the outer elongate shaft coaxially positioned over at least a portion of the inner elongate shaft;
a stent a stent having a proximal portion and a distal portion, the stent positioned on at least a portion of the inner elongate shaft;
a proximal constraining member releasably connected to the stent proximal portion;
a distal constraining member releasably connected to the stent distal portion;
holding the stent in a constrained configuration with torsional force applied to the stent by the proximal and distal constraining members the inner and outer elongate shafts in the first rotational position.
positioning the stent at the implant site;
expanding the stent to an expanded configuration by rotating the inner shaft relative to the outer shaft from the first rotational position to a second rotational position and releasing torsional force on the stent.

15. The method of claim 14, further comprising reapplying torsional force to the stent to move the stent from the expanded configuration to the constrained configuration by rotating the inner shaft relative to the outer shaft from the second rotational position to the first rotational position.

16. The method of claim 14, further comprising releasably connecting the distal constraining member to the inner shaft and releasably connecting the proximal constraining member to the outer shaft.

17. The method of claim 14, further comprising providing a removable sheath over the stent and a portion of the elongate shaft and withdrawing the sheath from the stent in the patient's lumen so that the stent is exposed in the constrained configuration.

18. The method of claim 14, further comprising providing a stiffening member extending into the lumen when the delivery device is in the patient's lumen.

19. The method of claim 14, further comprising providing a proximal retaining wire and a distal retaining wire to releasably lock the stent to the stent delivery system using the proximal and distal constraining members and allow the stent to move between the constrained configuration and the expanded configuration without release from the stent delivery system.

20. The method of claim 19, withdrawing the proximal and distal retaining wires to release the stent from the stent delivery system.
